(19) United States
(12) Patent Application Publication
(10) Pub. No.: US 2004/0157913 A1
(43) Pub. Date: Aug. 12, 2004

(54) Kappa opiate agonists for the treatment of bladder diseases

(76) Inventors: Jutta Jacob, Mainz (DE); Frank Weber, Dietzenbach (DE); Gerd Bartoszyk, Weiterstadt (DE); Christoph Seyfried, Seeheim (DE)

Correspondence Address: Millen White, Zelano & Branigan, Arlington Courthouse Plaza I, 2200 Clarendon Boulevard Suite 1400, Arlington, VA, 22201, US

(21) Appl. No.: 10/474,312

(22) PCT Filed: Mar. 13, 2002
(86) PCT No.: PCT/EP02/02756

(30) Foreign Application Priority Data
Apr. 5, 2001 (DE) 101 16 978.7

(51) Int. Cl. A61K 31/4015

(52) U.S. Cl. 514/424

(57) ABSTRACT

The invention relates to the use of a medicament n-methyl-N-[(1S)-1-phenyl-2((3S)-3-hydroxypyrrolidin-1-yl)ethyl]-2,2-diphenyl-acetamaide or one of the pharmacologically acceptable salts thereof for the production of medicament formulations for the treatment of bladder diseases, particularly irritable bladder syndrome and the pains associated therewith.

The invention relates to the use of the medicament N-methyl-N-[(1S)-1-phenyl-2-((3S)-3-hydroxypyrrolidin-1-yl)ethyl]-2,2-diphenylacetamide or one of its pharmacologically acceptable salts for the preparation of medicament formulations for the treatment of bladder diseases, in particular irritable bladder, and the pains associated therewith.

The medicament active ingredient N-methyl-N-[(1S)-1-phenyl-2-((3S)-3-hydroxypyrrolidin-1-yl)ethyl]-2,2-diphenylacetamide (asimadolin)

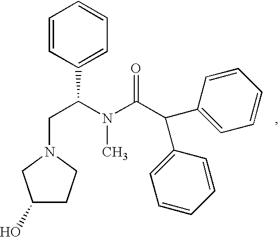

pharmacologically acceptable salts thereof and a process for the preparation are described in U.S. Pat. No. 5,532,266 (Example 1).

The medicament active ingredient N-methyl-N-[(1S)-1-phenyl-2-((3S)-3-hydroxypyrrolidin-1-yl)ethyl]-2,2-diphenylacetamide, in particular the hydrochloride thereof, has an analgesic, anti-inflammatory, antiasthmatic, diuretic, anticonvulsive, neuroprotective and antitussive action and, as a kappa-opiate agonist, is particularly suitable for the treatment of hyper-algesia caused by inflammation, for the treatment of cerebral oedema, in undersupply states (hypoxia), pain states, and for ameliorating secondary damage from ischaemia.

The use of N-methyl-N-[(1S)-1-phenyl-2-((3S)-3-hydroxypyrrolidin-1-yl)-ethyl]-2,2-diphenylacetamide or pharmacologically acceptable salts thereof for the preparation of a medicament for the treatment of inflammatory intestinal diseases and the disease symptoms associated therewith, for the treatment of severe pain, in particular of pain hypersensitivity occurring in back complaints, burn injuries, sunburn and rheumatic diseases, and for the treatment of postoperative pain and the ileus which frequently occurs after abdominal operations, are disclosed in EP 0 752 246.

N-Methyl-N-[(1S)-1-phenyl-2-((3S)-3-hydroxypyrrolidin-1-yl)ethyl]-2,2-diphenylacetamide or one of its pharmacologically acceptable salts is likewise suitable for the treatment of functional gastrointestinal diseases associated with pain and/or increased or reduced peristalsis, in particular of irritable bowel syndrome or for the treatment of non-ulcerative dyspepsia, obstipation, in particular opiode-induced obstipation, of arthritis, migraine, psoriasis or other itching skin diseases, dysmenorrhoea and fibromyalgia.

The object of the invention was to provide a pharmaceutically active compound which can be employed and is active in the treatment and/or prophylaxis of bladder diseases, in particular irritable bladder, also known as cytalgia, cystalgia, neuralgia vesicae or bladder neurosis, and which simultaneously ameliorate the pains associated with this disease and heal the disease.

The term irritable bladder is a chronic state of irritation of the lower urinary tract which occurs in particular in women. Symptoms are dysuria, imperative desire to urinate, pollakiuria, suprapubic and diffuse pain on sitting. There is frequently a pronounced discrepancy between subjective complaints and the objective findings. The most frequent causes are diseases of the psychovegetative or endocrine system. Irritable bladder should be distinguished from other syndromes, such as urinary tract infections and changes in the lower urinary tract, diseases of adjacent pelvic organs or central nervous system or spinal cord diseases (for example multiple sclerosis).

Surprisingly, it has been found that the medicament active ingredient N-methyl-N-[(1S)-1-phenyl-2-((3S)-3-hydroxypyrrolidin-1-yl)ethyl]-2,2-diphenylacetamide or one of its pharmacologically acceptable salts, in particular the hydrochloride, is surprisingly active for the treatment of bladder diseases, in particular irritable bladder, in spite of the known diuretic action of asimadolin.

The invention therefore relates to the use of the medicament N-methyl-N-[(1S)-1-phenyl-2-((3S)-3-hydroxypyrrolidin-1-yl)ethyl]-2,2-diphenylacetamide or one of its pharmacologically acceptable salts for the preparation of medicament formulations for the treatment of bladder diseases.

The invention therefore relates, in particular, to the use of the medicament N-methyl-N-[(1S)-1-phenyl-2-((3S)-3-hydroxypyrrolidin-1-yl)ethyl]-2,2-diphenylacetamide or one of its pharmacologically acceptable salts for the preparation of medicament formulations for the treatment of irritable bladder.

N-Methyl-N-[(1S)-1-phenyl-2-((3S)-3-hydroxypyrrolidin-1-yl)ethyl]-2,2-diphenylacetamide or one of its pharmacologically acceptable salts can therefore be used for the preparation of pharmaceutical preparations for the treatment of bladder diseases, in particular irritable bladder, by converting it into a suitable dosage form together with at least one excipient or adjuvant and, if desired, with one or more further active ingredients.

The invention therefore also relates to a pharmaceutical preparation characterised by a content of N-methyl-N-[(1S)-1-phenyl-2-((3S)-3-hydroxypyrrolidin-1-yl)ethyl]-2,2-diphenylacetamide or one of its pharmacologically acceptable salts for the treatment of bladder diseases.

The invention therefore also relates, in particular, to a pharmaceutical preparation characterised by a content of N-methyl-N-[(1S)-1-phenyl-2-((3S)-3-hydroxypyrrolidin-1-yl)ethyl]-2,2-diphenylacetamide or one of its pharmacologically acceptable salts for the treatment of irritable bladder.

The preparations obtained in this way can be employed as medicaments in human or veterinary medicine. Suitable excipient substances are organic or inorganic substances which are suitable for enteral (for example oral or rectal) or parenteral administration and which do not react with the novel compounds, for example water, vegetable oils, benzyl alcohols, polyethylene glycols, glycerol triacetate and other fatty acid glycerides, gelatine, soya lecithin, carbohydrates, such as lactose or starch, magnesium stearate, talc or cellulose.

Suitable for oral administration are, in particular, tablets, coated tablets, capsules, syrups, juices or drops. Of particular interest are lacquer-coated tablets and capsules having gastric-juice-resistant coatings or capsule shells. Suitable for rectal administration are suppositories, and suitable for parenteral administration are solutions, preferably oil-based or aqueous solutions, furthermore suspensions, emulsions or implants.

The active ingredients claimed in accordance with the invention may also be lyophilised and the resultant lyophilisates used, for example, for the preparation of injection preparations.

The stated preparations may be sterilised and/or comprise adjuvants, such as preservatives, stabilisers and/or wetting agents, emulsifiers, salts for modifying the osmotic pressure, buffer substances, colorants and/or flavours. If desired, they may also comprise one or more further active ingredients, for example one or more vitamins.

N-Methyl-N-[(1S)-1-phenyl-2-((3S)-3-hydroxypyrrolidin-1-yl)ethyl]-2,2-diphenylacetamide or one of its pharmacologically acceptable salts is generally administered analogously to other known preparations which are commercially available for the claimed indication, preferably in doses of between about 0.1 mg and 50 mg, in particular between 5 and 30 mg, per dosage unit. The daily dose is preferably between about 0.02 and 20 mg/kg, in particular 0.1 and 10 mg/kg of body weight.

However, the specific dose for each individual patient depends on a very wide variety of factors, for example on the efficacy of the specific compound employed, on the age, body weight, general state of health, sex, on the diet, on the time and method of administration, on the excretion rate, medicament combination and severity of the particular disease to which the therapy applies. Oral administration is preferred.

Animal models are described below which confirm the efficacy of asimadolin for the treatment of bladder diseases, in particular irritable bladder.

A model for measuring the effect on urine excretion is described in Lipschitz et al., J. Pharmacol. Exp. Ther. 1943; 79: 97-110. The substance to be investigated is given to rats who had previously been denied food overnight while being given free access to water. Increased urine excretion is provoked by simultaneous intraperitoneal injection of 100 ml/kg of physiological saline solution. Immediately after administration of the substance, the bladder was emptied by gentle massage of the abdomen above the bladder. The rats are subsequently kept in metabolism cages in which the urine is collected over a period of 6 hours. N-Methyl-N-[(1S)-1-phenyl-2-((3S)-3-hydroxypyrrolidin-1-yl)ethyl]-2,2-diphenylacetamide increases urine excretion as a function of dose, with twice the amount of urine being excreted at a dose of 100 mg/kg.

The effect on urine excretion in normal rats is tested analogously (i.e. without induction of increased urine excretion, see above). N-Methyl-N-[(1S)-1-phenyl-2-((3S)-3-hydroxypyrrolidin-1-yl)ethyl]-2,2-diphenylacetamide increases urine excretion as a function of dose, with 5.5 times the amount of urine being excreted at only 30 mg/kg po.

The classical animal model for irritable bladder is described in Ghoniem et al., Neurourol. Urodyn. 1995; 14: 657-65. In female apes, irritable bladder is induced by direct infusion of acetone into the bladder. The animals are kept in metabolism cages designed for continuous monitoring of miction (urination) of the animals. The frequency, emptying volumes and flow rate of the urine are measured continuously via urine flow meters. Comparison of urea absorption before and after acetone infusion shows that urea absorption is drastically increased after acetone infusion and only reaches the base value before acetone infusion again after four weeks. Furthermore, considerable changes in bladder physiology are observed in the first week after acetone infusion: the bladder performance, measured in ml/cm, drops by almost 95%. The emptying behaviour also changes considerably, with the frequency of emptying increasing greatly with the picture of frequent dribbling and at the same time with an emptying volume reduced by about 70%. Systematic observation of the behaviour of the animals over four weeks shows reduced frequency of general and in particular social activities as the behaviour repertoire, while stereotypical, self-directed behaviour patterns, such as self-grooming, scratching and fondling, increase considerably. These changes in behaviour observed in apes are consistent with the clinical picture of considerable discomfort and pain. N-Methyl-N-[(1S)-1-phenyl-2-((3S)-3-hydroxypyrrolidin-1-yl)ethyl]-2,2-diphenylacetamide in doses of 3, 10 and 30 mg/kg normalises bladder function in a dose-dependent manner.

1. Use of the medicament active ingredient N-methyl-N-[(1S)-1-phenyl-2-((3S)-3-hydroxypyrrolidin-1-yl)ethyl]-2,2-diphenylacetamide or one of its pharmacologically acceptable salts for the preparation of a medicament for the treatment of bladder diseases.

2. Pharmaceutical preparation, characterised by a content of at least N-methyl-N-[(1S)-1-phenyl-2-((3S)-3-hydroxypyrrolidin-1-yl)ethyl]-2,2-diphenylacetamide or one of its physiologically acceptable salts for the treatment of bladder diseases.

3. Use of the medicament active ingredient N-methyl-N-[(1S)-1-phenyl-2-((3S)-3-hydroxypyrrolidin-1-yl)ethyl]-2,2-diphenylacetamide or one of its pharmacologically acceptable salts for the preparation of a medicament for the treatment of irritable bladder.

4. Pharmaceutical preparation, characterised by a content of at least N-methyl-N-[(1S)-1-phenyl-2-((3S)-3-hydroxypyrrolidin-1-yl)ethyl]-2,2-diphenylacetamide or one of its physiologically acceptable salts for the treatment and/or prophylaxis of irritable bladder.
